SEQUENCE LISTING (14 sequences)

SEQ ID NO: 1 - DNA, 21 bp, Mus musculus
tgctcatcagcaagatccgag

SEQ ID NO: 2 - DNA, 21 bp, Mus musculus
ggaatggcaccatgttcacag

SEQ ID NO: 3 - DNA, 20 bp, Mus musculus
gaagccaacagcgaaaagac

SEQ ID NO: 4 - DNA, 20 bp, Mus musculus
cttgttttgtcatggagtcg

SEQ ID NO: 5 - DNA, 23 bp, Mus musculus
ctattttgtggtatggctggtgc

SEQ ID NO: 6 - DNA, 23 bp, Mus musculus
caagcattccagtggatgatgac

SEQ ID NO: 7 - DNA, 23 bp, Mus musculus
gtgctaaagctccagccccagac

SEQ ID NO: 8 - DNA, 22 bp, Mus musculus
agccagactctgtggcctcctg

SEQ ID NO: 9 - DNA, 21 bp, Mus musculus
agagatgagagaagaaaatgg

SEQ ID NO: 10 - DNA, 20 bp, Mus musculus
tgctcttgtaactttggatc

SEQ ID NO: 11 - DNA, 18 bp, Mus musculus
tggcagcacctggctgac

SEQ ID NO: 12 - DNA, 19 bp, Mus musculus
ggggcagtgtaacagagga

SEQ ID NO: 13 - DNA, 20 bp, Mus musculus
aaggtcaccatcacgggcac

SEQ ID NO: 14 - DNA, 20 bp, Mus musculus
tttaggttggcattctccac

(19) United States
(12) Patent Application Publication
(10) Pub. No.: US 2002/0048812 A1
(43) Pub. Date: Apr. 25, 2002

(54) Isolation and in vitro differentiation of conditionally immortalized murine olfactory receptor neurons

(76) Inventors: Gabriele V. Ronnett, Lutherville, MD (US); Robert Duncan Barber, Baltimore, MD (US); King-Wai Yau, Baltimore, MD (US)

Correspondence Address: BANNER & WITCOFF, 1001 G STREET N W, SUITE 1100, WASHINGTON, DC, 20001, US

(21) Appl. No.: 09/758,257

(22) Filed: Jan. 12, 2001

Related U.S. Application Data
(60) Provisional application No. 60/176,451, filed on Jan. 14, 2000.

(51) Int. Cl. C12N 5/06; C12N 5/08

(52) U.S. Cl. 435/368; 435/325

(57) ABSTRACT

Olfactory receptor cell lines are conditionally immortalized. Under permissive conditions they proliferate. Under nonpermissive conditions the cells differentiate into mature functional Olfactory Receptor Neurons (ORNs) expressing multiple olfactory neuron-specific markers. Exposure of cells of the clonal lines to a battery of odorants indicates a functionally heterogeneous population, in which approximately 1% of the cells respond to any particular single odorant. This heterogeneity suggests the potential of the cells of the cell line to express multiple different receptors and demonstrates that the cell line is an appropriate model for native Olfactory Receptor Neurons.

This application claims the benefit of provisional application Ser. No. 60/176,451 filed Jan. 14, 2000. The text of the provisional application is incorporated herein by reference.

This invention was made under grants DC02979 and EY06837 from the National Institutes of Health, an agency of the United States Government. Therefore, the government retains certain rights in the invention.

### TECHNICAL FIELD OF THE INVENTION

This invention is related to the area of olfaction. In particular it is related to cell lines useful in the testing of odorants for their physiological effects.

### BACKGROUND OF THE INVENTION

Several in vitro approaches have been used to address both odorant-coding and neuronal development issues in the olfactory system. These include receptor transfection studies (Raming et al., 1993; Krautwurst et al., 1998), primary cultures of olfactory epithelial slices (Gong et al., 1996) and primary cultures of ORNs dissociated by enzymatic digestion of olfactory epithelia (Calof and Chikaraishi, 1989; Ronnett et al., 1991; Vargas and Lucero, 1999). However, these studies have been hampered by inefficient odorant-receptor-protein translocation to the plasma membrane in heterologous systems (McClintock et al., 1997) or by limited survival of ORNs beyond seven days in culture (Ronnett et al., 1991; Vargas and Lucero, 1999). To overcome the survival issue, there have been several attempts to develop “immortal” olfactory cell lines.

Immortal olfactory receptor neuron cell lines fall into two categories. In one, immortalization occurred by chance (Wolozin et al., 1992; Vannelli et al., 1995), and in the other, immortalization occurred following planned manipulations (Largent et al., 1993; MacDonald et al., 1996). In the first category, cell lines were isolated from olfactory epithelia of adult human and aborted human foetuses. These cells expressed olfactory-specific proteins and gave functional responses to odorant stimulation in biochemical and fluorescence-based assays, respectively, (Wolozin et al., 1992; Vannelli et al., 1995). In the second category, immortalization was induced by one of two techniques. In the first technique, cells were immortalized by expression of the Simian Virus 40 large tumor antigen (TAg). Cells were cloned from a mouse in which the TAg was under the control of the regulatory elements of the olfactory marker protein gene (Largent et al., 1993). These cells expressed a growth-associated neuronal marker, and underwent morphological changes in response to “differentiating” agents. In a recent study, a conditionally-immortalized rat ORN cell line was isolated using transfected tsA58, a temperature-sensitive mutant of the TAg (Murrell and Hunter, 1999). Functional responses from transfected, exogenous odorant receptors could be observed in these cells, but single-cell cloning attempts were unsuccessful, and endogenous functional responses to odorants could not be demonstrated. In the second technique, the epithelium of bulbectomized adult mice was transfected with the immortalizing oncogene, n-myc (MacDonald et al., 1996). The presence of mRNA encoding olfactory markers was detected in these cells but for neither these cells nor the cells of Largent et al. (1993) were functional data available.

### SUMMARY OF THE INVENTION

It is an object of the present invention to provide a single cell-cloned, immortalized olfactory receptor neuronal cell line.

It is another object of the present invention to provide a method for making an olfactory cell line.

It is an object of the present invention to provide a method for inducing differentiation of cells.

It is still another object of the invention to provide a method for identifying cells which express receptors for odorant ligands.

Another object of the invention is to provide a method for determining compounds which induce differentiation of cells.

These and other objects of the invention are achieved by providing a single cell-cloned, immortalized olfactory receptor neuronal cell line, comprising cells which respond to odorant stimulation by increasing intracellular calcium concentration.

According to another aspect of the invention a method for inducing differentiation of cells of a single cell-cloned, immortalized olfactory receptor neuronal cell line comprising cells which respond to odorant stimulation by increasing intracellular calcium concentration is provided. Cells of the cell line are cultured at 37° C. in the absence of interferon-γ, whereby differentiation is induced.

According to another embodiment of the invention a method is provided for identifying cells which express receptors for odorant ligands. Cells of a single cell-cloned, immortalized olfactory receptor neuronal cell line comprising cells which respond to odorant stimulation by increasing intracellular calcium concentration are contacted with at least one odorant ligand. Calcium influx into the cells is observed to determine which cells of the cell line were induced by the odorant ligand to accumulate calcium.

Yet another embodiment of the invention is a method for determining compounds which induce differentiation of cells of a single cell-cloned, immortalized olfactory receptor neuronal cell line comprising cells which respond to odorant stimulation by increasing intracellular calcium concentration. The cells are contacted with a test compound. Differentiation of the cells is monitored. A test compound which induces differentiation is thereby identified.

The present invention thus provides the art with an important cell system in which to test compounds relating to olfactory reception and differentiation.

### BRIEF DESCRIPTION OF THE DRAWINGS

FIGS. 1A to 1H. Detection of NST, GFAP and NCAM in cells from permissive and non-permissive culture conditions.

FIGS. 1A-D. Cells from the heterogeneous cultures were labeled with primary antibodies for NST and GFAP and fluorophore-coupled secondary antibodies were used to visualize staining, rhodamine for NST and FITC for GFAP. Mutually-exclusive labeling for NST and GFAP was observed in cells grown both in permissive (FIGS. 1A and B) and non-permissive (FIGS. 1C and D) culture conditions.

FIGS. 1E-H. Cells were labeled as above with anti-NST and anti-NCAM antibodies. Co-localization of NST- and NCAM-labeling is observed in cells grown in both permissive (FIGS. 1E and F) and non-permissive (FIGS. 1G and H) culture conditions. The scale bar shown in A indicates 10 μm for each panel.

FIGS. 2A to 2C. Effects of Interferon, EGF, NGF, BDNF and NT-3 on the olfactory cell cultures.

FIG. 2A. Interferon causes a significant, dose-dependent increase in the number of NST+ cells in the heterogeneous culture, with an EC₅₀ of 0.4 U.ml⁻¹. Cells were incubated at 33° C. and were stained with anti-NST and rhodamine-coupled secondary antibodies seven days after plating. Cell counts were obtained from ten randomly selected areas in each experiment. Data were normalized to the mean number of NST+ cells in the absence of interferon and each point represents the mean ±S.E.M from three experiments.

FIG. 2B. EGF (•) and NGF (□) both have mitogenic effects in permissive conditions over and above the effect of interferon. NST+ cells were counted as described in (A) and were normalized to baseline levels in the presence of interferon (40 U.ml⁻¹). EC₅₀s of 2.9 ng.ml⁻¹ and 2.0 ng.ml⁻¹ were calculated for effects of EGF and NGF, respectively. Except for the presence of interferon (40 U.ml⁻¹), all experimental conditions were as in A.

FIG. 2C. Effects of EGF, NGF, BDNF and NT-3 on OMP expression as determined by Western blot. Cells were maintained in non-permissive conditions at 37° C. in the absence of growth factors (C) or in presence of EGF (E), NGF (N), BDNF (B) or NT-3 (3). Samples were isolated from the heterogeneous cell cultures and from olfactory tissue (T), and were separated on a SDS-polyacrylamide gel (15 μg protein per lane). OMP is a 19 kD protein and, in situ, is expressed only in mature olfactory receptor neurons. OMP was not detected in control cells (C), cultured in the absence of growth factors or in cells incubated in the presence of EGF. It was barely detectable from cells grown in the presence of NGF but in cells maintained in the presence of BDNF or NT-3, significant expression of OMP could be observed. Expression of OMP was greater from cells incubated with NT-3 than with BDNF and in both cases was less than the expression in tissue.

FIGS. 3A to 3J. Co-localization of olfactory-specific proteins with NST. Cells from the heterogeneous cultures were labeled with primary antibodies for NST, ACIII, Gα_{olf} and OMP. Fluorescent secondary antibodies were used to visualize staining, rhodamine for NST and FITC for Gα_{olf}, ACIII and OMP. Co-localization of Gα_{olf} and NST labeling was observed in both permissive (FIGS. 3A and B) and non-permissive (FIGS. 3C and D) culture conditions. Similarly, co-localization of ACIII and NST labeling was observed in cells grown in permissive (FIGS. 3E and F) and non-permissive (FIGS. 3G and H) conditions. OMP immunoreactivity (FIG. 3I) was only seen in cells maintained in non-permissive culture conditions where co-localization was observed with NST labeling (FIG. 3J). The scale bar shown in each panel indicates 10 μm for each pair of images.

FIG. 4. Amplification of olfactory-specific cDNAs with specific oligonucleotide primers.

mRNA from cells grown in permissive (33° C.) and non-permissive (37° C.) culture conditions and from tissue (T) was reverse transcribed (+) or incubated in the absence of enzyme (−). Specific oligonucleotide primers were then used to amplify the cDNA by PCR and the products were run on 2% agarose gels beside a molecular marker (M). The primer pairs used were specific for β-tubulin, the olfactory cyclic nucleotide-gated channel subunits 1 and 2 (OCNC1 and OCNC2), the Type III adenylate cyclase (ACIII), the olfactory-specific G protein alpha subunit, (Golf), a transcription factor in the olfactory epithelium (OE1), and olfactory marker protein (OMP). For each of the primer pairs tested, no product was obtained from reverse transcriptase-free controls. For all other conditions, with the exception OMP-amplification from cells in permissive conditions, specific products were obtained and confirmed by sequencing.

FIG. 5. Detection of large T antigen and olfactory-specific proteins by Western blot.

Samples were isolated from the heterogeneous cell cultures grown in permissive (33° C.) and non-permissive (37° C.) conditions and were separated on SDS-polyacrylamide gels (15 μg protein per lane). Antibodies were used against the large T antigen (TAg), the olfactory cyclic nucleotide-gated channel subunit 1 (OCNC1), olfactory marker protein (OMP), Type III adenylate cyclase (ACIII) and the olfactory-specific G protein alpha subunit (Gα_{olf}). TAg was only detected in cells cultured in permissive conditions and OMP was only detected in cells grown in non-permissive conditions. OCNC1, ACIII and Gα_{olf} were each detected in both cell extracts. Equal loading of the protein samples was confirmed by stripping and reprobing blots for monomeric actin.

FIGS. 6A to 6H. Clone, 3NA12, expresses markers of olfactory receptor neuron.

Cells from the clone 3NA12 were labeled with primary antibodies for NST, NCAM, NGFR (p75), OMP, NSE, ACIII and Gα_{olf}. Labeling was visualized with fluorescent secondary antibodies and in each panel, the scale bar represents 10 μm.

FIG. 6A and FIG. 6B. NST and NCAM staining, respectively, in cells cultured in permissive conditions.

Images are of the same cells and all cells except one are labeled with both antibodies. This cell (arrow) is only NCAM+ and did not stain NST+;

FIG. 6C. NST staining as described above for cells cultured in non-permissive conditions;

FIG. 6D. p75 NGF receptor staining for cells cultured in non-permissive conditions;

FIG. 6E. OMP staining for cells cultured in non-permissive conditions;

FIG. 6F. NSE staining for cells cultured in non-permissive conditions;

FIG. 6G. ACIII staining for cells cultured in non-permissive conditions; and,

FIG. 6H. Gα_{olf} staining for cells cultured in non-permissive conditions.

FIGS. 7A to 7F. Functional responses to odorants in the ORN clone, 3NA12.

Prior to experiments (2-4 days), cells were plated onto glass coverslips and incubated in permissive conditions. To prepare for stimulation, cells were loaded with Fura-2 (2 mM) for 30 minutes at 33° C., washed, and allowed to equilibrate for a further 30 minutes at room temperature. At the beginning of the experiment, cells were washed (W) to determine whether the mechanical disturbance associated with changing solution affected intracellular calcium concentrations. Odorants (isovaleric acid (I), heptaldehyde (H), acetophenone (A) and L-carvone (C), all 10 μM) were applied sequentially as a 5 ml bolus into the bath at time points indicated by the arrowheads. Responses to the four odorants can be seen in FIG. 7A-FIG. 7D, in which each trace represents data obtained from a single cell. Responses to two odorants by the same cell can be observed in panels B and D. Similar responses were observed in other experiments independent of the order of odorant application and in cells maintained in non-permissive culture conditions. Responses to repeated odorant applications are shown in FIGS. 7E and 7F.

FIG. 8. A schematic representation of the development of the olfactory receptor neuron.

The horizontal (HC) and the globose basal cells (GBC) are thought to be the precursors of cells in the olfactory epithelium. A mitotic cell (MC), possibly a GBC or its progeny, divides, resulting in the generation of a neuroblast (Nb). The neuroblast then develops into an immature receptor neuron (IRN) and eventually into a mature olfactory receptor neuron (ORN) accompanied by migration of the cell body upwards through the olfactory epithelium. Indicated below the diagram is a correlated time course of antigen expression. Full lines indicate confirmed tissue expression and dashed lines indicate an unclear onset of expression. From our experiments, it is suggested that cells in permissive and non-permissive conditions may be equivalent to cells in the olfactory epithelium as indicated. The points at which EGF, NGF, BDNF and NT-3 may act are also depicted.

### DETAILED DESCRIPTION

To develop a method that reproducibly yields clonal lines of murine odorant receptor neurons (ORNs), the inventors have generated immortal cell cultures from the H-2K^{b}-tsA58 transgenic mouse (Jat et al., 1991, available from Jackson Laboratories, Bar Harbor, Me.). The genome of this mouse harbors the γ-interferon-inducible mouse major histocompatibility complex promoter sequence, situated upstream of the temperature-sensitive TAg. In situ, this transgene is inactive but, when cells are isolated from this mouse and cultured in the presence of γ-interferon and at 33° C. (permissive conditions), the cells exhibit a conditional immortalization. In non-permissive conditions (i.e., at 37° C. in the absence of interferon) the cells differentiate (Chambers et al., 1993; Barber et al., 1997). Therefore, this mouse provides an ideal source for the generation of ORN cell lines. The inventors have found that clonal ORN cell lines can be isolated from this mouse, and they can be maintained and passaged in permissive culture conditions. Moreover, the inventors have found that odorant-stimulated responses can be observed in these cells and phenotypic changes consistent with cell differentiation occur in non-permissive culture conditions. These cells can be used to study ORN development and permit the efficient, functional expression of exogenous odorant receptors.

The inventors have created fifty-six independent, single cell-cloned, immortalized olfactory receptor neuronal cell lines which comprise cells which respond to odorant stimulation. One of these cell lines has been extensively characterized and studied. Cell lines of the invention undergo differentiation at 37° C. in the absence of interferon-γ. This is due to the inactivation and transcriptional deinduction of the T antigen. While the particular cell lines which were obtained in the course of the experimental work of the inventors is derived from a mouse, similar cell lines can be derived from other rodent and mammalian transgenic animals. Preferably the cell line contains one or two alleles of H-2K^{b}-tsA58, which is an allele of SV40 large T antigen which encodes a temperature sensitive protein. The allele is under the control of a γ-interferon-inducible mouse major histocompatibility complex promoter sequence. More preferably the cell line is derived from olfactory epithelium. Cells of the cell line can be transfected with an expression construct encoding an odorant receptor. Thus certain odorant receptors can be overexpressed or regulatably expressed in an appropriate cellular background to facilitate determination of the properties of odorant receptors and how they work together. Similarly, one can use such transfected cell lines to determine how different receptors work together to recognize odorants combinatorially. Thus the cell lines provide an ideal system in which currently cryptic receptors can be identified.

According to the present invention one can cause the cells of the cell line to differentiate by culturing the cells at 37° C. in the absence of interferon-γ. Under such conditions the cells differentiate and express olfactory marker protein (OMP). Either or both of these conditions can be applied to effect differentiation. Other agents can be added to induced differentiate either alone or in combination with the non-permissive conditions mentioned. These include brain-derived neurotrophic factor (BDNF) and neurotrophin-3 (NT-3).

As discussed in more detail below, cell lines can be made by obtaining olfactory epithelia. Preferably the olfactory epithelia is from a transgenic mouse comprising H-2K^{b}-tsA58, which is an allele of SV40 large T antigen. The allele encodes a temperature sensitive protein is under the control of an interferon-γ-inducible mouse major histocompatibility complex promoter sequence. Alternatively, another means of immortalizing the cells can be used, such as another oncogene, or inactivating a tumor suppressor gene, or using a dominant negative allele of a tumor suppressor gene. The other means of immortalization may or may not be conditional. The olfactory epithelia can be disrupted to form a suspension comprising single cells. This can be done mechanically and/or enzymatically. Single cells so produced can be cultured at 33° C. in the presence of interferon-γ. Preferably the cells will be passaged repeatedly as single cells to form a single cell-cloned cell line.

Maintaining the conditional cell lines of the present invention in the undifferentiated state has been found to be facilitated by culturing them in the presence of epidermal growth factor (EGF) and neuronal growth factor (NGF). Differentiation of cells of a cell line according to the invention can be accomplished by culturing the cells of at 37° C. and/or in the absence of interferon-γ. The former effects the T antigen's stability and the latter prevents its transcriptional induction. In addition, at least one factor selected from the group consisting of brain-derived neurotrophic factor (BDNF) and neurotrophin-3 (NT-3) can be added to the cells to enhance induction of differentiation. One of the key indications of differentiation of an olfactory receptor neuronal cell is the expression of olfactory marker protein (OMP).

The cell lines of the present invention can be used to identify cells which express receptors for odorant ligands. Cells can be contacted with one or more odorant ligands. Calcium influx into the cells can be observed or measured to determine which cells of the cell line were induced by the odorant ligand to accumulate calcium. This can be determined by measuring an increase in intracellular calcium concentration. Odorant stimulation can also be measured electrophysiologically, as is well known in the art. Cells which express an odorant receptor can be used to isolate the receptor. For example, reverse transcription-polymerase chain reaction (RT-PCR) can be used to amplify and ultimately sequence the gene which encodes the expressed odorant receptor. If more than one receptor is expressed in the cells which are stimulated by the odorant, they may combinatorially recognize the odorant ligand. Such tests can also be done with mixtures of two or more odorants.

The cells of the present invention can be used to identify compounds which induce differentiation of olfactory receptor neuronal cells. Test compounds can be contacted with the cells and the cells monitored to determine differentiation of the cells. Any standard measure of differentiation can be used, including but not limited to observing morphology of the cells and determining expression of protein markers of mature neuronal cells. One important marker of mature neuronal olfactory receptor cells is olfactory marker protein or OMP.

The above disclosure generally describes the present invention. A more complete understanding can be obtained by reference to the following specific examples which are provided herein for purposes of illustration only, and are not intended to limit the scope of the invention.

### EXAMPLE 1

Immortalization of Olfactory Receptor Neurons in Culture

Cell cultures were obtained from all ages of heterozygous H-2K^{b}-tsA58 transgenic mice. The proliferation/survival rate was greatest when cells were isolated from early post-natal animals by mechanical dissociation of the epithelia. The optimum basal culture medium was established as Minimum Essential Medium with L-valine replaced by D-valine (MDV) and supplemented with non-dialyzed FBS. Provisionally, EGF (20 ng.ml⁻¹) and 2.5 S NGF (10 ng.ml⁻¹) were added to the culture medium to enhance proliferation/survival. Further quantitative characterization of the effects of NGF and EGF was undertaken later (see below) after the presence of neurons in the cell cultures had been confirmed. Laminin was found to be the most permissive substrate.

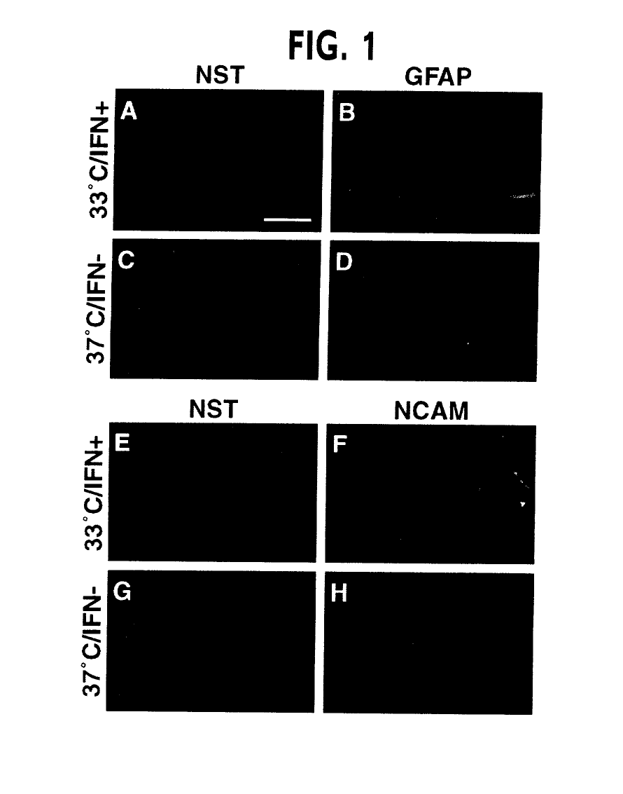

Mutually exclusive labeling of cells by NST and GFAP antibodies was observed in both permissive (33° C., +interferon) and non-permissive (37° C., −interferon) culture conditions in double-labeling experiments (FIGS. 1A-D). Some cells were not stained with either antibody and were not investigated further. The presence of neurons in both permissive and non-permissive culture conditions was confirmed by NCAM labeling. Co-localization, though not absolute overlap, of NST (FIGS. 1E, G) and NCAM (FIGS. 1F, H) immunoreactivity was observed through multiple passages and provided further evidence for the existence of a neuronal population. The GFAP+ population of cells decreased through multiple passages, with no cells stained with anti-GFAP antibodies by passage 5 in either culture condition (data not shown). This may be due to the inability of glia to adhere efficiently to laminin-coated culture dishes (Ronnett et al., 1991).

In the experiments described here, we have defined a reliable method for the production of conditionally-immortal ORNs from the H-2K^{b}-tsA58 transgenic mouse. This method has enabled us to generate many clonal ORN cell lines that express olfactory markers and demonstrate a functional response to odorants. Cells are proliferative when the immortalizing SV40 TAg is expressed, and protein-expression patterns consistent with mature ORNs can be induced when the SV40 TAg is degraded/absent. Immortalization occurs as a result of TAg binding to the retinoblastoma protein (Rb) and p53. Usually, in non-proliferating cells, growth factors are bound by Rb and p53 acts as a brake on DNA transcription. However, when these two proteins are bound by TAg, their functions are disrupted. Consequently, transcription can proceed and cell immortalization can occur, as observed in our permissive culture conditions. In non-permissive conditions (37 □C), the TAg is degraded and cell differentiation begins to occur. In addition to the TAg-mediated immortalization, the survival of our olfactory receptor neuron cultures may in part stem from the fact that neurogenesis naturally occurs post-natally in the olfactory epithelium. Consistent with this idea, hippocampal granule cells, which have been shown to divide in situ in marmoset monkeys (Gould et al., 1998), can be conditionally immortalized from fetal H-2K^{b}-sA58 mice (Kershaw et al., 1994). Likewise, the non-neuronal cells that have been immortalized from the H-2K^{b}-tsA58 mouse are proliferative in situ (Whitehead et al., 1993).

Characterization of the Olfactory Receptor Neurons and a Model for Development of the Olfactory Epithelium

Cells in the heterogeneous cultures of the H-2K^{b}-tsA58 mouse olfactory epithelium express both neuronal and olfactory markers. All of the olfactory markers that we tested, except OMP, were present in cells cultured in both permissive and non-permissive conditions. OMP was found only in cells maintained in non-permissive culture conditions, which may be indicative of induction of a more differentiated phenotype (Margolis, 1988). This is illustrated in FIG. 8 in which schematic diagram depicting a model for ORN development is shown together with the approximate time courses of expression of various proteins. In this model, precursor cells (thought to be globose basal cells; Caggiano et al., 1994), give rise to daughter cells that develop into mature olfactory receptor neurons. As indicated in FIG. 8, the development of mature olfactory receptor neurons can be described immunochemically by the expression of different proteins. For example, NST expression occurs early in development but begins to wane in the most mature olfactory receptor neurons (Roskams et al., 1998). Conversely, OMP expression is restricted to mature olfactory receptor neurons (Margolis, 1988), and Gα_{olf} and ACIII appear to be expressed in both immature and mature neurons. Phenomena broadly similar to these have been observed in our cultures: In permissive culture conditions, all cells that express Gα_{olf} or ACIII also express NST, but not vice versa. By contrast, in non-permissive culture conditions, not all cells that express Gα_{olf}, ACIII or OMP express NST. Therefore, inactivation of the TAg may be associated with the induction of olfactory receptor neuron maturation in the heterogeneous culture.

Based on these patterns of labeling, probable developmental windows encompassing our cultures are shown in FIG. 8.

### EXAMPLE 2

Optimization of the Cell Culture Conditions

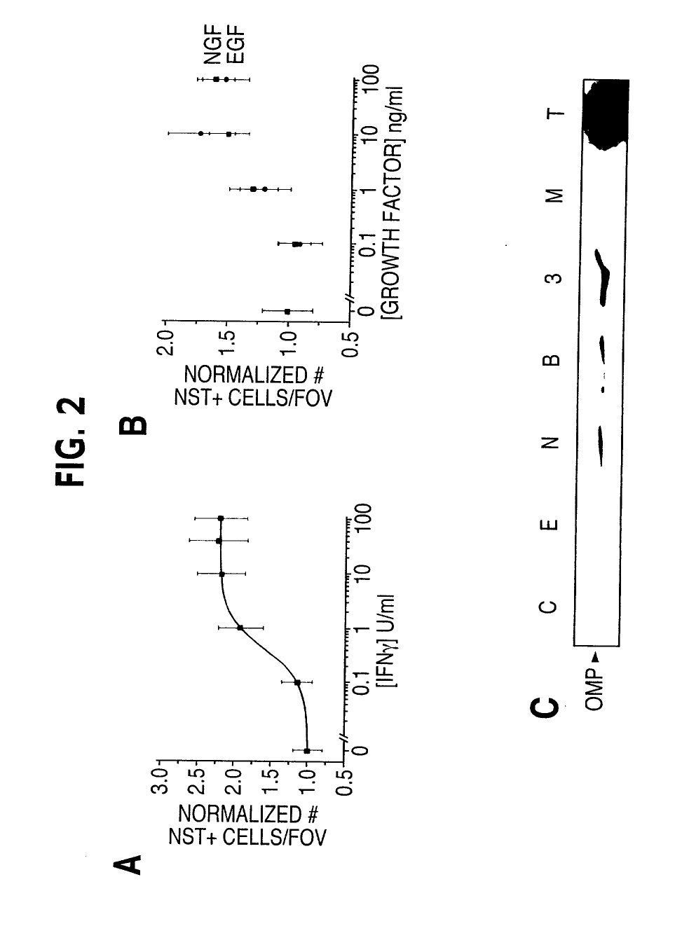

To determine the optimal conditions for cell survival, growth, differentiation and maturation, cells were cultured in permissive and non-permissive conditions in the presence of interferon, EGF, NGF, BDNF and NT-3. These data are summarized in Table 1 and are shown in FIG. 2. In permissive conditions, interferon caused a significant increase in the number of NST+ cells, with a half-maximal effective concentration (EC₅₀) of 0.4 U.ml⁻¹ (FIG. 2A). Likewise, in permissive conditions, EGF or NGF increased the number of NST+ cells (EC₅₀s of 2.0±1.4 ng.ml⁻¹ and 2.9±1.9 ng.ml⁻¹, respectively, see FIG. 2B), but BDNF or NT-3 had no effect (Table 1). The effects of EGF, NGF, BDNF and NT-3 on cell maturation in non-permissive culture conditions were

TABLE 1
The effects of different growth factors in permissive and
non-permissive culture conditions.

| | |
|---|---|
| 33° C. | 37° C. |

| | Normalized | | Normalized | |
|---|---|---|---|---|
| | # of | | # of | |
| | NST+ Cells | EC₅₀ | NST+ Cells | OMP Levels |
| Control | 1.0 ± 0.2 | | 1.0 ± 0.1 | — |
| Interferon | 2.0 ± 0.2* | 0.4 U.ml⁻¹ | 1.9 ± 0.1* | ND |
| 40 U.ml⁻¹) | | | | |
| EGF | 1.7 ± 0.2* | 2.0 ng.ml⁻¹ | ND | +/− |
| (10 ng.ml⁻¹) | | | | |
| NGF | 1.5 ± 0.2* | 2.9 ng.ml⁻¹ | ND | +++ |
| (100 ng.ml⁻¹) | | | | |
| BDNF | 1.3 ± 0.2 | NC | 1.3 ± 0.2 | +++ |
| (100 ng.ml⁻¹) | | | | |
| NT-3 | 0.8 ± 0.1 | NC | 1.4 ± 0.1* | ++++++ |
| (100 ng.ml⁻¹) | | | | |
| The effects of different growth factors were assessed by counting cells labeled with the anti-NST antibody. For experiments with interferon, cells were grown in the presence of increasing interferon concentrations and were normalized to the number of NST+ cells cultured in the absence of interferon. For EGF, NGF, BDNF and NT-3, all experiments were conducted in the presence of 40 U.ml⁻¹ interferon and the data were normalized to the number of NST+ cells at that point. | | | | |
| # Determination of statistical significance (*, P < 0.05) was made using one-way ANOVA tests. EC₅₀s were calculated from sigmoidal curves fitted to the data. “NC” indicates that the EC₅₀ was not calculated as no significant effect was observed. OMP levels were estimated from the Western blot, shown in FIG. 2C. “—” indicates that no expression could be observed, “ND” indicates no data and the number of “+” symbols corresponds | | | | |
| # approximately to the level of OMP detected. | | | | |

assayed by Western blot detection of olfactory marker protein (OMP), a marker of mature olfactory receptor neurons (Margolis, 1988). OMP was most readily detected in cells incubated with NT-3 and, to a lesser degree, was detected in cells incubated with BDNF or NGF (Table 1 and FIG. 2C). Much weaker OMP immunoreactivity could also be detected in cells grown in the presence of EGF but not in cells maintained in the absence of any growth factors (FIG. 2C).

Based on these data, in all subsequent experiments, interferon (10 U.ml⁻¹), EGF (10 ng.ml⁻¹) and NGF (10 ng.ml⁻¹) were added to the culture medium in permissive conditions to enhance proliferation/survival, and BDNF (20 ng.ml⁻¹) and NT-3 (10 ng.ml⁻¹) were added to the medium in non-permissive culture conditions to promote maturation.

Optimization of Culture Conditions

The data presented here have shown that NGF and EGF enhance the proliferation or survival of the cultured cells in permissive culture conditions, and that BDNF and NT-3 act as differentiating factors. In situ, NGF is produced in the olfactory bulb and is transported to the olfactory epithelium (Miwa et al., 1998). NGF receptors have been detected immunohistochemically in the rat and human olfactory epithelium (Balboni et al., 1991; Aiba et al., 1993) and are up-regulated following olfactory nerve transection (Miwa et al., 1993). It has also been shown in tissue culture studies that NGF increases ORN survival and neurite extension (Ronnett et al., 1991) but has no effect on cell division as measured by radiolabeled DNA precursor uptake (Farbman and Buchholz, 1996). In the present study, NGF enhanced proliferation/survival of our cultures and it was shown that the p75 NGF receptor was expressed in the clonal cells 3NA12.

These data support the hypothesis that NGF and its associated receptors promote survival of the ORN (Aiba et al., 1993).

EGF and the EGF-family member TGFα are both potent mitogens in the olfactory epithelium, acting on the basal cells that give rise to the ORNs (Mahanthappa and Schwarting, 1993; Farbman and Buchholz, 1996). EGF receptor mRNA and protein have been detected in the olfactory epithelium and have been localized to the basal cell layer (Krishna et al., 1996; Balboni et al., 1991). Of each of the factors we tested, EGF stimulated the greatest increase of NST+ cells in our heterogeneous cultures in permissive conditions. These are data that support a neurogenic role for the EGF family in the olfactory epithelium (see FIG. 8).

Consistent with mitogenic/survival roles for EGF and NGF, these factors did not have a dramatic effect on cellular maturation. Potential candidates for maturation or differentiating factors include BDNF and NT-3. Expression of BDNF has been detected in the granule cell layer of the olfactory bulb (Guthrie and Gall, 1991) and in the basal cell layer of the olfactory epithelium (Buckland and Cunningham, 1998). BDNF has been shown to promote survival in mouse olfactory epithelium (Holcomb et al., 1995) and the TrkB receptor is expressed by ORNs (Holcomb et al., 1995; Deckner et al., 1993). The TrkC receptor is selectively expressed in mature ORNs, and mRNA encoding NT-3 has been stated to be present in the olfactory epithelium (Roskams et al., 1996). In the present study, BDNF and NT-3 were shown to enhance cellular progression into a “mature/differentiated” phenotype in non-permissive culture conditions (FIG. 2C), as determined by OMP expression. In the olfactory epithelium, neuronal precursors are TrkA-positive, become TrkB-positive following mitosis and eventually become TrkC-positive as mature neurons (Roskams et al., 1996). The topographical distribution of these receptors is consistent with a role for the TrkB receptor in stimulating both survival and differentiation of ORNs and for the TrkC receptor in stimulating or maintaining maturation. This hypothesis is supported by the data described here, in which both BDNF and NT-3 enhance the differentiation/maturation state of the cells (see FIG. 8). However, since only four factors have been studied in these experiments, the data are almost certainly incomplete if the whole epithelium in situ is to be considered. The fibroblast growth factor family has been proposed to have mitogenic, proliferative and phenotypic effects on olfactory receptor neurons (DeHamer et al., 1994; Goldstein et al., 1997; MacDonald et al., 1996) as have ciliary neurotrophic factor, leukemia-inhibitory factor, interleukin-6 and retinoic acid (Farbman, 1994; Plendl et al., 1999). It is also possibly that the growth factors that have been investigated in this and previous studies have had an indirect or paracrine effect on the ORNs. These issues can be examined for the first time using the clonal cell lines.

### EXAMPLE 3

Olfactory Receptor Neurons are Detected by Immunocytochemistry

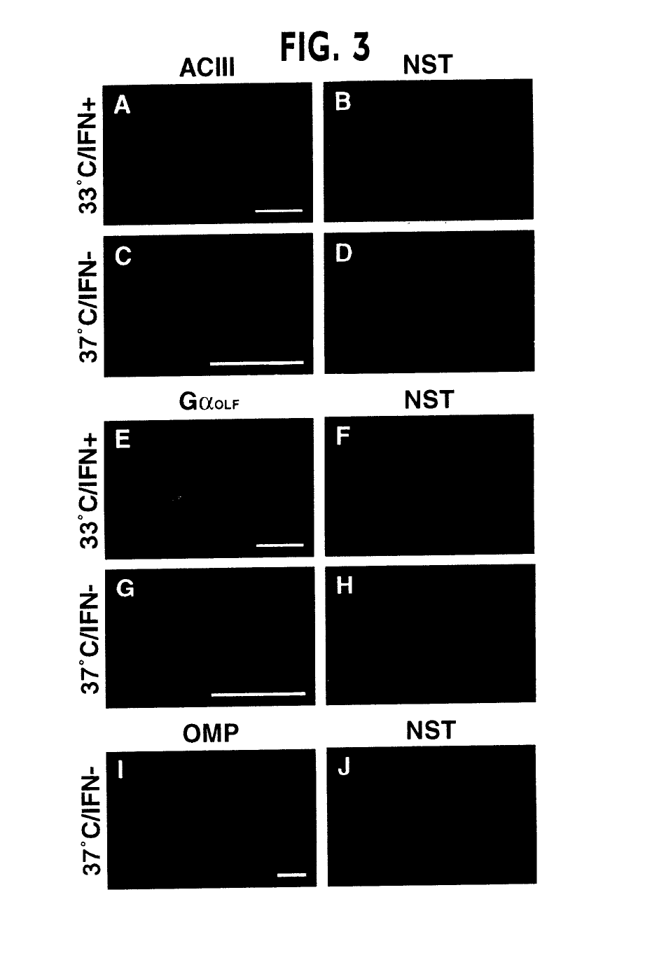

To demonstrate whether the neurons detected in this culture system were olfactory in nature, the presence of three olfactory-specific markers was investigated (FIG. 3). Immunoreactivity for Gα_{olf} and ACII, two components of the olfactory second-messenger cascade (Jones and Reed, 1989; Bakalyar and Reed, 1990), could be observed in both permissive and non-permissive conditions (FIGS. 3A & C for permissive and FIGS. 3E & G for non-permissive conditions, with corresponding co-localization of NST expression shown in FIGS. 3B, D, F and H). OMP was not detected in permissive culture conditions (data not shown), but was detected in the BDNF/NT-3-supplemented, non-permissive conditions (FIG. 3I, with FIG. 3J showing co-localization of NST). These data indicate that olfactory receptor neurons are present in our cultures. In addition to the double-labeled cells, some cells in non-permissive conditions cells stained only with OMP+, Gα_{olf}+ and ACIII+ and did not label with NST. This observation is consistent with the staining of NST in the olfactory epithelium, which has been shown to wane in the very mature neurons (Roskams et al., 1998).

### EXAMPLE 4

Olfactory-specific mRNA is Detected by RT-PCR

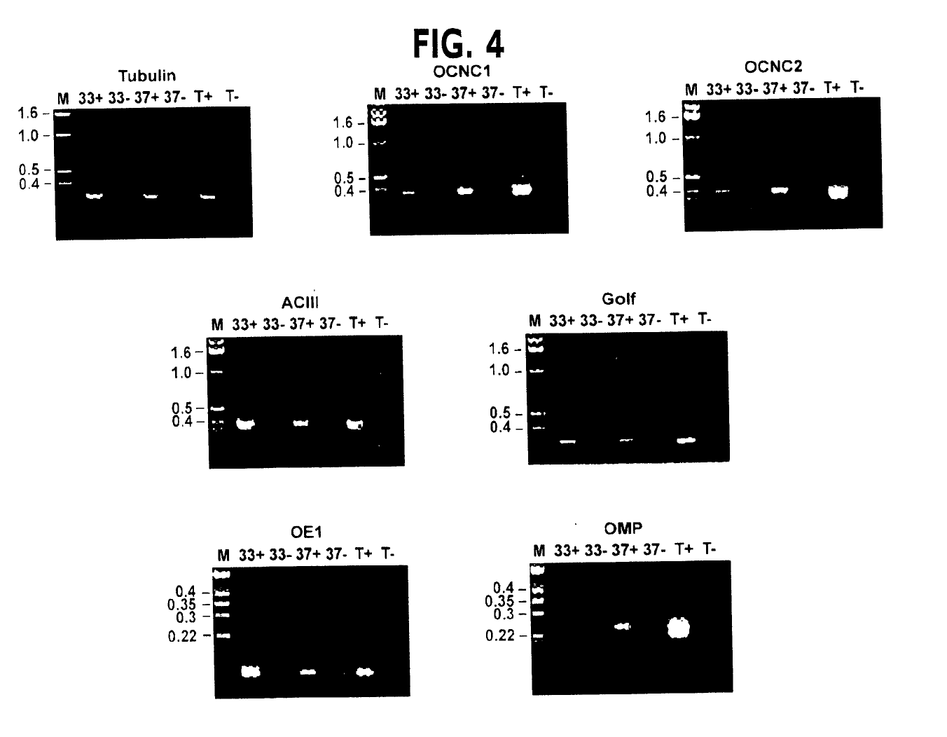

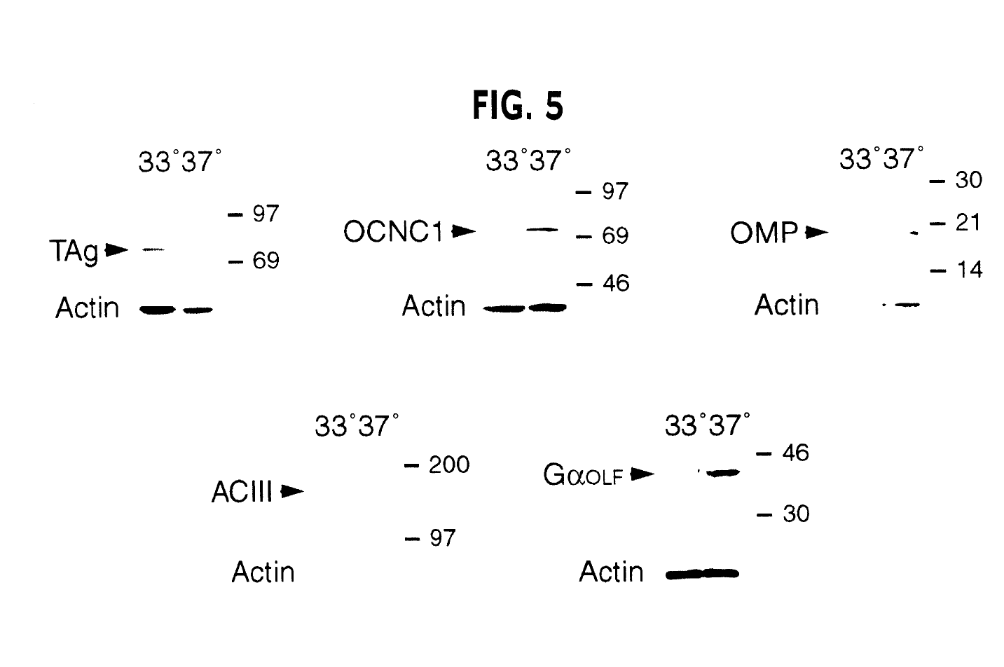

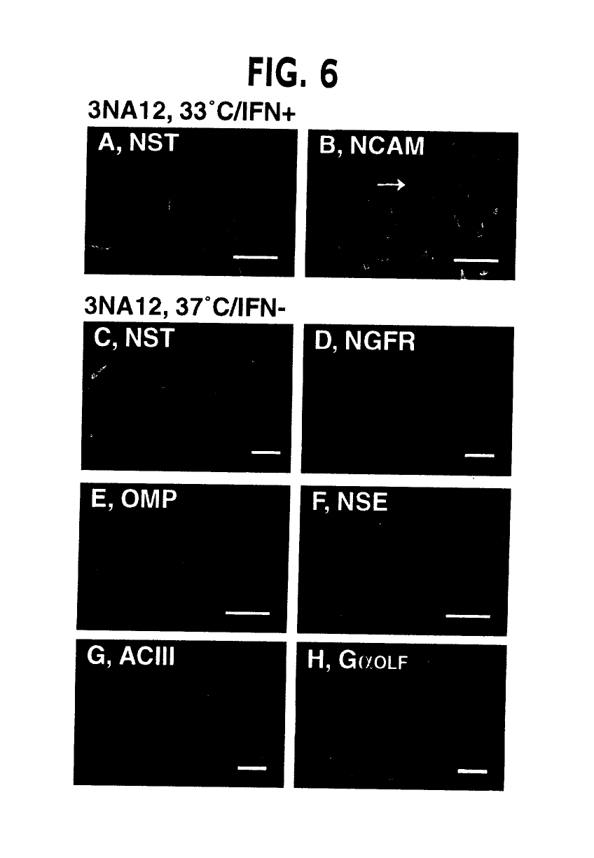

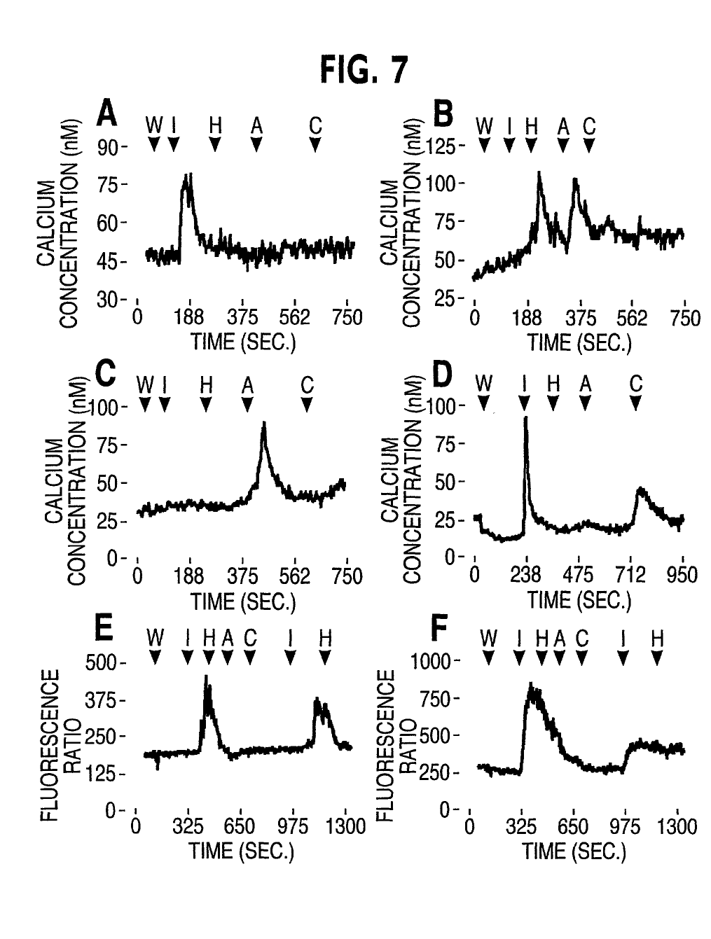

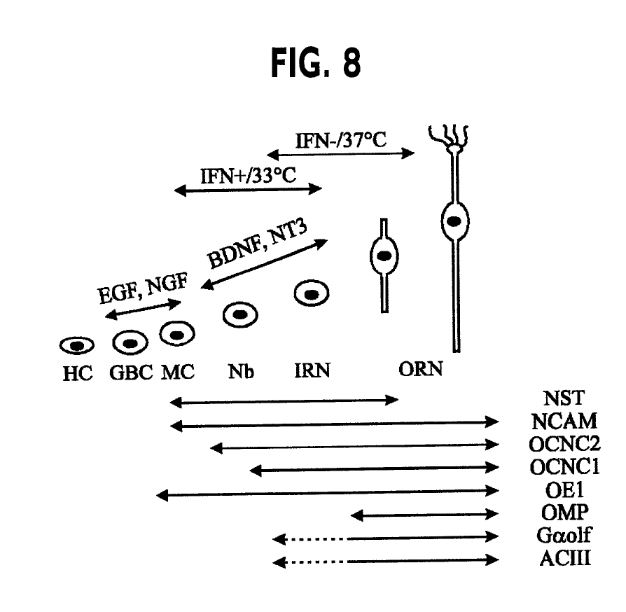

mRNA was isolated from the olfactory epithelia of early post-natal heterozygous mice and from cells cultured in permissive or non-permissive conditions. Following reverse transcription of the mRNA, specific primers for β-tubulin (as a positive control), the olfactory cyclic nucleotide-gated channel subunits OCNC1 and OCNC2, Gα_{olf}, ACIII, OE1 (an olfactory transcription factor) and OMP were used to amplify the cDNA by PCR. The products had the expected sizes (FIG. 4) and their identities were confirmed by sequencing. To determine the origin of the PCR products, the primers for the two OCNC subunits and OE1 were designed across introns. The sizes of the products from genomic DNA contaminants would have been 0.6, 1.2 and 0.8 kb for OCNC1, OCNC2 and OE1, respectively, rather than the 0.36, 0.38 and 0.1 kb products obtained. mRNA for each of the olfactory markers tested, with the exception of OMP, was present in cells cultured in permissive conditions. In cells maintained in non-permissive culture conditions, mRNA for each of the markers, including OMP, was detected. These data are consistent with the presence of olfactory receptor neurons in the cultures and a process of cellular maturation/differentiation when cells are switched from permissive to non-permissive culture conditions.

### EXAMPLE 5

T Antigen and Olfactory-specific Proteins are Detected by Western Blot

Further confirmation of the data obtained by immunocytochemistry and PCR analysis was obtained from Western blots. Whole-cell extracts were probed for TAg and for several olfactory-specific markers (FIG. 5). The TAg ran at ˜70 kD and was only expressed in cells cultured in permissive conditions (FIG. 5A), consistent with the theoretical expectation of TAg expression in cells isolated from the H-2K^{b}-tsA58 transgenic mouse. The presence of the olfactory proteins OCNC1, ACIII, Gα_{olf} and OMP was demonstrated by the bands at the expected sizes of c. 76 kD, 170 kD, 44 kD and 19 kD, respectively. The band intensities of the olfactory proteins for cells maintained in non-permissive culture conditions were at least equivalent, if not higher, than those in permissive conditions, even though sample loading was equivalent (as judged by the intensity of actin labeling). Most notable was OMP, which was only expressed in cells grown in non-permissive conditions, confirming the immunocytochemical and PCR results. The presence of OE1 was also investigated and several bands at different molecular weights were observed (data not shown). These may reflect the presence of the different OE transcription factor isoforms or the recognition of proteins arising from multiple transcription start sites within the OE1 gene (S. Wang and R. Reed, personal communication). These data, together with the immunocytochemistry and the RT-PCR results, indicate that differentiation or maturation of the cells may occur with the change in culture conditions and inactivation of the TAg.

### EXAMPLE 6

Derivation of Clonal Cell Lines by Fluorescence-activated Cell (FAC) Sorting

It was shown earlier (FIGS. 1G-J) that NCAM was expressed in the heterogeneous culture system and that it co-localized with NST. Cells were labeled with the anti-NCAM antibody and attempts were made to isolate independent clones by fluorescence-activated cell sorting (data not shown). After labeling, a marked shift of the fluorescence scatter distribution was observed when compared to controls. One hundred and forty-four NCAM+ cells were plated singly into 96-well plates and were cultured in permissive conditions. Thirty-nine proliferative colonies were isolated, expanded and were stored in liquid nitrogen. All attempts at recovery of frozen clonal lines have been successful. One of these clonal lines, 3NA12, has been characterized further. NST, NCAM, NSE and the p75 NGFR, as well as the olfactory markers Gα_{olf}, ACIII and OMP, were detected in 3NA12 cells (FIGS. 6A-H). Neuronal and olfactory marker co-localization was confirmed in double-labeling experiments (data not shown).

### EXAMPLE 7

Responses to Odorants in a Clonal Line are Observed by Calcium Imaging

To determine whether cells from the 3NA12 clone were capable of functional responses to odorants, single odorants were applied to cells loaded with Fura-2. Preliminary experiments indicated that responses could be observed in cells from both permissive and non-permissive culture conditions (data not shown). Since the onset of odorant receptor expression is at c. E13 in the rat (Strotmann et al., 1995), and each of the components of the signal transduction pathway is present in cells cultured in permissive conditions, cells at permissive conditions were chosen for all subsequent experiments. Four odorants (acetophenone, heptaldehyde, isovaleric acid and L-carvone) were applied sequentially to a total of 1256 cells in nineteen experiments. In two experiments (139 cells) no cells responded. In the other seventeen experiments, odorant application stimulated an increase in intracellular calcium concentration in 54 cells, the equivalent of 4.3% of cells responding (see FIGS. 7A-D for examples).

Of the responsive cells, six cells responded to two odorants but no cells responded to more than two odorants. An even distribution of responses to each of the different odorants was observed: 15 cells responded to acetophenone, 17 to heptaldehyde, 12 to isovaleric acid and 16 to L-carvone, approximately equal to 1% of cells responding to each odorant. Repeat applications of the odorants stimulated the cells to respond again, generally with a decrease in the signal observed (see FIGS. 7E & F for examples). Control experiments were carried out with primary cultures of mouse olfactory bulb neurons and rat ORNs. Both groups of cells were exposed to three odorant mixtures, each containing four odorants. No changes in intracellular calcium concentration were observed following odorant application in 780 olfactory bulb neurons in twelve experiments. In the primary cultured rat ORNs, responses to the odorant mixtures were seen in 12 out of 387 cells in six experiments. The frequency of responses in the rat ORN cultures was equivalent to 0.25% of cells responding per odorant, a factor of approximately four-fold less than 3NA 12 cells.

Functional Responses and Odorant Receptor Expression

Another application for these clonal cell lines relates to the control of odorant receptor expression. Upon stimulation with odorants, increases in the intracellular calcium concentration can be measured in the clonal cell line, 3NA12. This calcium rise is presumably a result of an elevation of cAMP concentration which, in turn, opens the calcium-permeable, olfactory cyclic nucleotide-gated channels (Leinders-Zufall et al., 1998; Kurahashi and Shibuya, 1990). This suggests that some level of endogenous odorant receptor expression occurs in 3NA12 cells and that the cells can transduce odorant/receptor interactions. This indicates that the signal transduction machinery is not only present in these cells but is also functionally intact.

Both the percentage of 3NA12 cells responding to odors and the profiles of the responses in individual cells suggests that multiple odorant receptors may be expressed in the 3NA12 clonal cell line. Four individual, structurally-diverse odorants were applied to 3NA12 cells and functional responses to each odorant were observed in consistent, low percentages of cells. Among responsive cells, 90% of cells were stimulated by a single odorant while 10% of cells responded to two odorants. The sum of the frequency of these responses was similar to the frequency of responses to a mixture of the same odorants in the primary cultures of dissociated rat ORNs. Since it is expected that the odorant receptors expressed by primary cultures of ORNs reflect a cross-section of the total olfactory receptor population, our results suggest that a relatively broad spectrum of odorant receptors may be expressed by 3NA12 cells. The same conclusion can be derived from the response profiles of the cells. Responses were observed to each of the structurally-diverse odorants tested, and the most direct explanation for this observation is that the cells in the 3NA12 cell line express different odorant receptors. Consequently, it can be proposed that the control of receptor expression in 3NA12 cells is dependent upon extrinsic stimuli and is not determined entirely at a genetic level, as might be expected of clonal cells. This hypothesis can now be tested using the model system we have developed.

The low frequency of responses of the 3NA12 clone may also make these cells ideal for heterologous odorant receptor expression. In the past, receptor expression studies have been hampered by the lack of satisfactory heterologous expression systems in which odorant receptor proteins can be efficiently translocated to the plasma membrane. Since the 3NA12 cells are capable of functional responses and retain other differentiated features of olfactory receptor neurons, they are likely to be able to target exogenous olfactory receptors to the plasma membrane effectively and to signal odorant-induced receptor activation.

### EXAMPLE 8

Materials and Methods

Cell Culture

To keep the amount of expressed TAg to the minimum required for immortalization (Noble et al., 1995), heterozygous offspring from homozygous male H-2K^{b}-tsA58 transgenic and C57B1 female mice (Charles River Laboratories, Wilmington Mass., and Jackson Laboratory, Bar Harbor, Me., respectively) were used to generate the conditionally immortal neuronal cell culture. Cells were isolated in a procedure modified from a previously established technique (Ronnett et al., 1991). Briefly, post-natal day 1-3 animals were decapitated and the heads were sectioned longitudinally. The olfactory epithelium and nasal septum were removed and placed in an Eppendorf tube containing 500 μl culture medium. The culture medium consisted of Minimum Essential Medium in which the L-valine has been replaced by D-valine (MDV). This was supplemented with 10% fetal bovine serum (FBS), 4 mM glutamine, kanamycin (100 μg.ml⁻¹), gentamicin (50 U.ml⁻¹) and amphotericin B (2.5 μg.ml⁻¹; all from Gibco BRL, Gaithersburg, Md.). The MDV medium was chosen because fibroblasts lack D-amino acid oxidase and the reduction of L-valine inhibits fibroblast survival and growth (Gilbert et al., 1986). Tissue was centrifuged at low speed for two minutes and then most of the medium was removed. The cell pellet was chopped briefly with a pair of fine-point scissors before being resuspended in supplemented cell culture medium. The resultant cell clumps and cell suspension were plated out onto 2- or 4-chamber glass slides (Nunc, Naperville, Ill.) or plastic dishes (Fisher Scientific, Pittsburgh, Pa.). To enhance ORN adhesion, all cell culture materials were pretreated with laminin (0.125 mg.ml⁻¹ in serum-free media, Collaborative Biomedical Products, Bedford, Mass.) for at least 12 hours, prior to plating. Cells were maintained in culture at 33° C. (the permissive temperature for the SV40 TAg) and, to stimulate transcription of the transgene, the culture medium was supplemented with murine γ-interferon (40 U.ml⁻¹, Genzyme, Cambridge, Mass.). Provisionally, in permissive conditions, the cell culture medium was supplemented with epidermal growth factor (EGF, 20 ng.ml⁻¹, Gibco BRL) and 2.5 S nerve growth factor (NGF, 10 ng.ml⁻¹, Gibco BRL) before thorough characterizations of their effects were made in later experiments.

To determine optimum conditions for cell survival, growth, differentiation or maturation, numerous combinations of culture conditions were tested. These included i) removal of the olfactory epithelium from newborn, 4-week-old, and sexually mature animals, ii) subjecting tissue to digestion by trypsin (Gibco BRL, 0.5 g.l⁻¹ for 60 minutes at 37° C.), iii) addition of growth factors to the culture medium and iv) the use of diverse culture media. Cells were plated out and fed in each of the following media: Neurobasal/B27, Neurobasal/B27/serum, MDV/dialyzed serum and MDV/FBS (all Gibco BRL), all in the presence of IFN (40 U.ml⁻¹), EGF (20 ng.ml⁻¹) and NGF (10 ng.ml⁻¹). Subsequently, the effects of different concentrations of interferon, EGF, NGF, brain-derived neurotrophic factor (BDNF, Peprotech, Rocky Hill, N.J.) and neurotrophin-3 (NT-3, Peprotech) were also tested. Based on those experiments, interferon (10 U.ml⁻¹), EGF (10 ng.ml⁻¹) and NGF (10 ng.ml⁻¹) were routinely added to the culture medium for all cells maintained in permissive culture conditions (33° C.).

Cells were always maintained in permissive culture conditions except when attempts were made to obtain a differentiated phenotype through transgene inactivation. In this case, cells were incubated in non-permissive culture conditions, i.e. at 37° C. in γ-interferon-free media for seven days, before the effects of transgene inactivation were determined. The effects of EGF, NGF, BDNF and NT-3 in promoting cell maturation/differentiation were assessed and, based on those experiments, BDNF and NT-3 were routinely added to the culture medium for cells in non-permissive culture conditions. In both culture environments, the air was kept humidified and contained 5% CO₂. Cells from homozygous C57B1 mice were used as controls and did not survive beyond a few days.

Immunocytochemistry

Cells were rinsed in phosphate buffered saline (PBS, pH 7.4, at 37° C.), and fixed in either ice-cold methanol (10 minutes at −20° C.) or fresh paraformaldehyde (2% in PBS for 20 minutes on ice). After fixation, cells were permeabilized by incubation with 0.1% Triton X100 in PBS for 20 minutes on ice. Cells were rinsed (3×5 minutes, PBS) and blocked with 10% normal donkey serum (NDS, Jackson Immunoresearch Inc., West Grove, Pa.) for 60 minutes. Incubation with primary antibodies was carried out in 5% NDS in PBS at 4° C. overnight. The following primary antibodies and dilutions were used: anti-neuron-specific tubulin (NST, 1:1000, BAbCo, Richmond, Calif.), anti-glial fibrillary acidic protein (GFAP, 1:800, Dako Corporation, Carpinteria, Calif.), anti-neural cell adhesion molecule (NCAM, 1:100, Chemicon, Temecula, Calif.), anti-Gα_{olf}, (1:1000), anti-adenylate cyclase type III (ACIII, 1:1000, both Santa Cruz Biotechnology, Santa Cruz, Calif.), anti-OE1, a transcription factor in the olfactory epithelium (1:1000, gift from R. Reed, Johns Hopkins University School of Medicine, Department of Molecular Biology and Genetics), anti-OMP (1:1000, gift from F. Margolis, University of Maryland Medical School), anti-neuron-specific enolase (NSE, 1:2, Incstar Corp., Stillwater, Minn.) and anti-p75 NGF receptor (p75 NGFR, 1:1000, Boehringer Mannheim Corporation, Indianapolis, Ind.). The following day, cells were rinsed (3×5 minutes) with PBS and incubated sequentially with the appropriate fluorescein- or rhodamine-coupled secondary antibodies (1:50 and 1:100 respectively) in 5% NDS in PBS for 60 minutes. Subsequently, cells were rinsed and were mounted in Aquamount (VWR, West Chester, Pa.) and photographed using 100 ASA color Kodak Ektachrome film.

PCR Analysis

The polymerase chain reaction was used to confirm the presence of olfactory-specific markers in the cells obtained from the H-2K^{b}-tsA58 transgenic mouse. Using the MicroFast Track Kit (Invitrogen, Carlsbad, Calif.), mRNA was isolated from cell cultures in both permissive and non-permissive conditions and from olfactory tissue (postnatal day 2). cDNA was produced from the mRNA using Superscript Reverse Transcriptase II (Gibco) with a mixture of random hexamer and oligo dT primers. Enzyme-free reactions were carried out as controls for the following PCR analysis. The resulting cDNA was amplified by PCR using the Expand High Fidelity PCR System (Boehringer Mannheim Corp.) with primers for the following markers:

Tubulin, 45° C., 5′-TGCTCATCAGCAAGATCCGAG (SEQ ID NO:1); 3′-GGAATGGCACCATGTTCACAG (SEQ ID NO:2),

OE1, 54° C., 5′ -GAAGCCAACAGCGAAAAGAC (SEQ ID NO:3); 3′-CTTGTTTTGTCATGGAGTCG (SEQ ID NO:4),

OCNC1, 56° C., 5′-CTATTTTGTGGTATGGCTGGTGC (SEQ ID NO:5); 3′-CAAGCATTCCAGTGGATGATGAC (SEQ ID NO:6),

OCNC2, 65° C., 5′-GTGCTAAAGCTCCAGCCCCAGAC (SEQ ID NO:7); 3′-AGCCAGACTCTGTGGCCTCCTG(SEQ ID NO:8),

Gα_{olf}, 50° C., 5′ -AGAGATGAGAGAAGAAAATGG (SEQ ID NO:9); 3′-TGCTCTTGTAACTTTGGATC(SEQ ID NO:10),

ACIII, 56° C., 5′ -TGGCAGCACCTGGCTGAC (SEQ ID NO:11); 3′-GGGGCAGTGTAACAGAGGA(SEQ ID NO:12) and

OMP, 60° C., 5′ -AAGGTCACCATCACGGGCAC (SEQ ID NO:13); 3′-TTTAGGTTGGCATTCTCCAC(SEQ ID NO:14).

The amplification protocol was 94° C.×5 minutes, (94° C.×1 minute, 6° C.×1 minute, 72° C.×1 minute)×35 cycles, and 72° C.×10 minutes final extension, where is the primer-specific annealing temperature listed above. Products were ligated into the pCR2.1 vector supplied with the TA Cloning Kit (Invitrogen) and transformed. Colonies were screened by PCR and positive clones were grown up as mini-preps for plasmid isolation (Qiagen, Valencia, Calif.). The cDNAs obtained were sequenced in the HHMI Sequencing Laboratory to confirm the presence of the correct insert. All protocols were carried out according to the manufacturers' instructions.

Western Blot Analysis

Cells were scraped into a cold isolation buffer containing 20 mM Na(N-tris[hydroxymethyl]methyl-2-aminoethanesulfonic acid) (Na TES), 10 mM mannitol and 1% Triton X-100 at pH 7.4. The isolation buffer was supplemented with phenylmethylsulfonyl fluoride (30 μg.ml⁻¹), leupeptin (2 μg.ml⁻¹), benzamidine (16 μg.ml^{'1}), pepstatin (2 μg.ml⁻¹), and lima bean trypsin inhibitor (50 μg.ml⁻¹) to prevent protease activity. After five freeze/thaw cycles, cell extracts were centrifuged at 2,000 g for 5 minutes to pellet nuclei and cell debris. Supernatants were assayed for protein concentration using the Bicinchoninic Acid Protein Reagent kit (Pierce Chemical Co., Rockford, Ill.).

Samples from cell extracts were prepared for electrophoresis by boiling for 5 minutes in 125 mM Tris loading buffer (pH 6.8) containing 1% SDS and 3.2% β-mercaptoethanol. Proteins (15 μg per lane) were separated on SDS-polyacrylamide gels containing 6% acrylamide (0.19% N,N′-methylene-bisacrylamide), 10% acrylamide (0.27% N,N′-methylene-bisacrylamide) or 16.5 % acrylamide (0.67% N,N′-methylene-bisacrylamide) alongside molecular weight standards (Amersham Corp., Arlington Heights, Ill.). Proteins were then transferred to Immobilon-P membranes (Millipore, Bedford, Mass.) in 25 mM Tris, 200 mM glycine (pH 8.5), 20% methanol for 1.5 hours at 500 mA. Blots were blocked with 5% non-fat dry milk diluted in 50 mM Tris HCl, 150 mM NaCl, pH 7.5, containing 0.05% Tween-20 (TTBS) for up to 45 minutes. Subsequently, blots were incubated in TTBS with the appropriate dilution of primary antibody for 2 hours at room temperature. In order to probe the transferred proteins, the following antisera were used at the indicated dilutions: polyclonal rabbit antisera against OCNC1 (Bradley et al., 1997), (1:1000); AC III, Gα_{olf} (1:500 and 1:1000 respectively); polyclonal goat antisera against OMP (1:2500); monoclonal mouse ascites against the large T Antigen (Gift from T. Kelly, Johns Hopkins University School of Medicine, Department of Molecular Biology and Genetics, 1:500). After washing, blots were incubated for 45 minutes with HRP-conjugated donkey anti-rabbit IgG (1:10,000, Amersham Corp.), HRP-conjugated rabbit anti-mouse IgG (1:5000, Amersham Corp.) or HRP-conjugated donkey anti-goat IgG antibodies (1:10,000, Jackson Immunoresearch Inc.) and visualized using the Enhanced Chemiluminescence kit (Amersham Corp.). Exposure times ranged from 1 to 10 minutes. Blots were then stripped by incubation for 20 minutes at 50° C. in 62.5 mM Tris HCl, pH 6.7, 2% SDS, and 100 mM β-mercaptoethanol in a shaking water bath and reprobed with monoclonal mouse hybridoma media against monomeric actin (JLA20, Developmental Studies Hybridoma Bank, 1:500) to control for protein loading. Visualization was achieved as described above.

Fluorescence-activated Cell Sorting

Passage 3 cells from one 100 mm plate were subjected to a brief enzymatic (trypsin) digestion prior to antibody labeling and fluorescence-activated cell sorting. Cells were incubated sequentially with a rabbit anti-NCAM antibody and with a FITC-coupled donkey anti-rabbit secondary antibody for 45 minutes each. Negative-control and background experiments (2×60 mm dishes) were carried out by omitting either both labeling steps or the primary labeling step, respectively. All incubations were carried out on ice in the presence of 10% normal donkey serum. Cell sorting was carried out on a FACStarPLUS, modified with Turbosort (Becton Dickinson, Bedford, Mass.), and gating was based on fluorescence intensity, subject to cell size (forward scatter) and granularity (side scatter) restrictions. Single cells were transferred into laminin-coated 96-well plates and maintained in culture as described previously.

Subsequently, cells were passaged into single wells of a 12 well plate and then into a 10 cm dish prior to storage in liquid nitrogen. Clones have been named on the basis of the well from which they were obtained. For example, clone 3NA12 was obtained from well A12 in the third (3) plate which contained cells sorted on the basis of NCAM-immunoreactivity.

Calcium Imaging

Cloned olfactory receptor neurons were plated onto glass coverslips up to one week before imaging. On the day of analysis, the culture medium was removed and replaced with MDV containing 2 μM Fura-2-AM (Molecular Probes, Eugene, Oreg.) and 0.2% Pluronic F-127 (Sigma) dissolved in DMSO. Cells were incubated at appropriate culture temperatures (33° C. or 37° C.) for at least 30 minutes, rinsed and allowed to equilibrate in medium for 30 minutes prior to experiments. Additionally, to ensure that a response could not be induced mechanically, each experiment was started by washing the cells with bath solution in the same manner used in odorant application. For solution exchange, 5 ml of bath solution or odorant test solution was pipeffed manually into the bath (volume, 300 μl) fitted with a continual suction pump. This ensured complete replacement of the bath solution with the incoming solution without altering bath volume. Single odorants were applied to the cloned cell line 3NA12 and mixtures of odorants were applied to primary cultures of mouse olfactory bulb neurons and rat ORNs. The mixtures consisted of: Citronellal, Pinene, Geraniol, N-amyl acetate (Mixture 1); Acetophenone, Heptaldehyde, Isovaleric acid, L-Carvone (Mixture 2); and Ethyl vanillin, Helional, Isoamyl acetate, Cineole (Mixture 3). When single odorants were used, they were taken from Mixture 2. Stock solutions of odorants (20 mM in DMSO) were made up every second day and diluted 1:2000 in bath solution (final concentration, 10 μM each) within seconds of application. The bath solution contained 140 mM NaCl, 5 mM KCl, 2 mM CaCl₂, 10 mM HEPES and 10 mM glucose at pH 7.4. Calcium imaging was performed as described previously (Krautwurst et al., 1998; Grynkiewicz et al., 1985), in which ratiometric measurements were obtained using the Zeiss/Attofluor-Ratiovision imaging system on a Zeiss Axiovert 135 microscope fitted with an F Fluor 20×/1.30 lens. Cells were illuminated at 340 nm and 380 nm and the emission at 510 nm was monitored using an intensified CCD camera. The Attofluor-Ratiovision software was used to derive the Ca²⁺-dependent ratio. Solutions of CaCl₂ (1 mM) and EDTA (1 mM) containing Fura-2 pentasodium salt (10 mM, Molecular Probes) were used to provide a two-point calibration of the experimental set-up as directed by the instrument manufacturer (Atto Instruments, Rockville, Md.).

1. A single cell-cloned, immortalized olfactory receptor neuronal cell line, comprising cells which respond to odorant stimulation by increasing intracellular calcium concentration.

2. The cell line of claim 1 which comprises cells which are capable of differentiating.

3. The cell line of claim 1 which is derived from a mouse.

4. The cell line of claim 3 wherein said mouse is heterozygous for H-2K^{b}-tsA58, which is an allele of SV40 large T antigen which encodes a temperature sensitive protein, wherein said allele is under the control of a γ-interferon-inducible mouse major histocompatibility complex promoter sequence.

5. The cell line of claim 4 which is derived from olfactory epithelium.

6. The cell line of claim 1 which is transfected with an expression construct encoding an odorant receptor.

7. A culture of cells of the cell line of claim 4 which has been cultured at 37° C. in the absence of interferon-γ such that the cells express olfactory marker protein (OMP).

8. A method for making a cell line according to claim 4 comprising:
obtaining olfactory epithelia from a transgenic mouse comprising H-2K^{b}-tsA58, which is an allele of SV40 large T antigen which encodes a temperature sensitive protein, wherein said allele is under the control of a interferon-γ-inducible mouse major histocompatibility complex promoter sequence;
disrupting the olfactory epithelia to form a suspension comprising single cells;
culturing the single cells at 33° C. in the presence of interferon-γ, whereby a cell line is formed which is stimulatable by odorants and is capable of differentiating into mature olfactory receptor neuronal cells.

9. The method of claim 8 further comprising the step of passaging single cells to form a single cell-cloned cell line.

10. The method of claim 8 wherein the step of disrupting is performed by mechanical means without enzymatic disruption.

11. The method of claim 8 wherein the single cells are cultured in the presence of epidermal growth factor (EGF) and neuronal growth factor (NGF).

12. A method for inducing differentiation of cells of a cell line according to claim 4 comprising:
culturing the cells of claim 4 at 37° C. in the absence of interferon-γ.

13. A method for inducing differentiation of cells of a cell line according to claim 1 comprising:
culturing said cells with at least one factor selected from the group consisting of brain-derived neurotrophic factor (BDNF) and neurotrophin-3 (NT-3).

14. The method of claim 12 wherein said cell line is derived from a mouse which is heterozygous for H-2K^{b}-tsA58.

15. A method for identifying cells which express receptors for odorant ligands, comprising:
contacting cells of a cell line according to claim 1 with at least one odorant ligand;
measuring odorant stimulation of the cells to determine which cells of the cell line were stimulated by the odorant ligand.

16. The method of claim 15 wherein odorant stimulation is measured by observing calcium influx into the cell.

17. The method of claim 15 wherein two or more odorants are contacted with the cells.

18. The method of claim 15 wherein said cell line is derived from a mouse which is heterozygous for H-2K^{b}-tsA58.

19. The method of claim 18 wherein the cells are maintained at 33° C. in the presence of interferon-γ.

20. The method of claim 18 wherein the cells are maintained at 37° C. in the absence of interferon-γ.

21. The method of claim 15 further comprising the step of determining which odorant receptors are expressed by the cell or cells which are stimulated by the odorant ligand.

22. The method of claim 21 wherein the expression of an odorant receptor is determined using reverse transcription-polymerase chain reaction (RT-PCR).

23. A method for determining compounds which induce differentiation of cells of the cell line of claim 1, comprising:
contacting cells of the cell line of claim 1 with a test compound;
monitoring differentiation of the cells in response to the test compound.

24. The method of claim 23 wherein the step of monitoring is performed by observing morphology of the cells.

25. The method of claim 23 wherein the step of monitoring is performed by determining expression of protein markers of mature neuronal cells.
